(19) United States
(12) Patent Application Publication
(10) Pub. No.: US 2022/0260561 A1
(43) Pub. Date: Aug. 18, 2022

(54) DEVICES AND METHODS FOR RAPID SCREENING OF DRUGS OF ABUSE AND OTHER ANALYTES

(71) Applicant: THE TRUSTEES OF THE UNIVERSITY OF PENNSYLVANIA, Philadelphia, PA (US)

(72) Inventors: Ping WANG, Wynnewood, PA (US); Zhao LI, Beijing (CN); Hui CHEN, Malden, MA (US); Keng-Ku LIU, Hsinchu City (TW)

(21) Appl. No.: 17/627,260

(22) PCT Filed: Jul. 13, 2020
(86) PCT No.: PCT/US2020/041777

§ 371 Date: Jan. 14, 2022

Related U.S. Application Data
(60) Provisional application No. 62/874,643, filed on Jul. 16, 2019.

(51) Int. Cl. G01N 33/543; G01N 33/94

(52) U.S. Cl. CPC G01N 33/54388; G01N 33/9486; G01N 33/54346

(57) ABSTRACT

Provided are devices, kits, and methods for rapid detection of analytes of interest, such as drugs of abuse, at comparatively low concentrations. The technology includes competitive assay lateral flow devices that utilize a nanoparticle-antibody complex to provide a visually-perceptible marker upon contact with a sample having above a cutoff level of analyte.

### RELATED APPLICATIONS

The present application claims priority to and the benefit of U.S. patent application No. 62/874,643, “Devices And Methods For Rapid Screening Of Drugs Of Abuse And Other Analytes” (filed Jul. 16, 2019), the entirety of which application is incorporated herein by reference for any and all purposes.

### TECHNICAL FIELD

The present disclosure pertains to the field of analyte detection and to the field of lateral flow assays.

### BACKGROUND

There is a long-felt need for rapid screening of analytes, including such analytes as opioids and other drugs of abuse. Existing screening techniques, however, are deficient in speed and/or sensitivity and can, in some cases, require operation by personnel trained in operation of medical diagnostics (as opposed to medical treatment providers). Accordingly, there is a long-felt need for improved screening devices and methods.

### SUMMARY

In meeting the described long-felt needs, the present disclosure provides screening devices for screening a sample for an analyte, comprising: a pervious medium, the pervious medium comprising a test region and a control region; the test region comprising a conjugate of the analyte immobilized to the test region of the pervious medium, the control region comprising a control binding partner immobilized to the control region of the pervious medium, the control binding partner being complementary to a detection complex that comprises (i) a nanoparticle and (ii) a detection binding partner that is complementary to the analyte, and the test region (a) being visually perceptible following contact with a testing sample formed from at least the detection complex and a sample originally comprising the analyte at less than a cutoff concentration, and (b) being visually imperceptible following contact with a testing sample formed from at least the detection complex and a sample originally comprising the analyte at greater than a cutoff concentration.

Also provided are screening devices for screening a sample, comprising: a pervious medium, the pervious medium comprising a test region and a control region; the test region comprising a conjugate of an analyte immobilized to the pervious medium, the control region comprising a control binding partner immobilized to the pervious medium, the control binding partner being complementary to a detection complex that comprises (i) a nanoparticle and (ii) a detection partner complementary to the analyte of interest, and wherein the test region comprises a visually perceptible level of the detection complex following contact with a sample formed from at least the detection complex and a sample originally comprising less than about 1 ng/mL of the analyte.

Also provided are screening methods, comprising: contacting a sample with an amount of a detection complex, the detection complex comprising a (i) nanoparticle and (ii) a detection partner complementary to an analyte, the contacting giving rise to a treated sample; introducing the treated sample to a pervious medium, the pervious medium comprising a test region and a control region, the test region comprising a conjugate of the analyte immobilized to the test region of the pervious medium, the control region comprising a control binding partner immobilized to the control region of the pervious medium, wherein the amount of the detection complex is selected such that the test region is (a) visually perceptible following contact with a testing sample formed from at least the detection complex and a sample originally comprising the analyte at less than a cutoff concentration, and (b) visually imperceptible following contact with a testing sample formed from at least the detection complex and a sample originally comprising the analyte at greater than a cutoff concentration.

Additionally provided are kits, comprising: (i) a screening device for screening a sample for an analyte, comprising: a pervious medium, the pervious medium comprising a test region and a control region; the test region comprising a conjugate of the analyte immobilized to the test region of the pervious medium, the control region comprising a control binding partner immobilized to the control region of the pervious medium, the control binding partner being complementary to a detection complex that comprises (1) a nanoparticle and (2) a detection binding partner that is complementary to the analyte, and the test region (a) being visually perceptible following contact with a testing sample formed from at least the detection complex and a sample originally comprising the analyte at less than a cutoff concentration, and (b) being visually imperceptible following contact with a testing sample formed from at least the detection complex and a sample originally comprising the analyte at greater than a cutoff concentration; and (ii) a supply of the detection complex.

### BRIEF DESCRIPTION OF THE DRAWINGS

The file of this patent or application contains at least one drawing/photograph executed in color. Copies of this patent or patent application publication with color drawing(s)/photograph(s) will be provided by the Office upon request and payment of the necessary fee.

In the drawings, which are not necessarily drawn to scale, like numerals can describe similar components in different views. Like numerals having different letter suffixes can represent different instances of similar components. The drawings illustrate generally, by way of example, but not by way of limitation, various aspects discussed in the present document. In the drawings:

FIG. 1 provides a depiction (not to scale) of an exemplary fentanyl lateral flow strip based on a competitive lateral flow immunoassay.

FIGS. 2A-2D illustrate the analytical sensitivity and specificity of the lateral flow strip. FIG. 2A provides Images of strips tested with different concentration (ng/mL) of fentanyl, norfentanyl and carfentanyl in artificial urine. Dotted rectangles indicate areas scanned in FIG. 2B. FIG. 2B provides densitometry results of areas marked by dotted rectangles. FIG. 2C provides normalized signal intensity (Test/Control) at different fentanyl concentration, 10 ng/mL norfentanyl (red star) and 1000 ng/mL carfentanyl (blue triangle). All experiments were repeated three times independently. FIG. 2D provides an illustration of on-strip testing of common drugs of abuse, treatment drugs and known interfering drugs with other immunoassays. AMP: amphetamine, COC: cocaine, MET: methadone, BUP: buprenorphine, MOR: morphine, THC: tetrahydrocannabinol, NAL: naloxone, ACT: acetaminophen. All concentration units are ng/mL.

FIG. 3 provides a STARD diagram of the consecutive ED patient group.

FIG. 4 provides individual results of clinical urine samples tested using the fentanyl strip and the LC-MS/MS method. Y-axis is fentanyl equivalent concentration as determined by the LC-MS/MS method, calculated as (LC-MS/MS fentanyl+LC-MS/MS norfentanyl×0.08).

FIG. 5A provides an exemplary transmission electron microscopy image of the synthetic AuNPs. Scale bar, 100 nm. The inset in the corner shows the enlarged view of AuNPs. Scale bar, 50 nm. FIG. 5B provides an exemplary UV-visible extinction spectrum of the unconjugated AuNPs (black curve) and antibody modified AuNPs (red curve). AuNPs and Ab-AuNPs displayed the extinction maximum at 520 nm and 528 nm, respectively.

FIG. 6 provides an exemplary illustration of Ab-AuNP conjugate concentration. Different dilutions of Ab-AuNP conjugate (10×, 50×, 80×, and 100×) were tested. The concentration of fentanyl was 0, 1, and 10 ng/mL in each set of experiments.

FIG. 7 provides exemplary mages of LFA strips tested with different concentrations of norfentanyl (0, 10, 15, 30, 50, and 80 ng/mL).

FIG. 8 provides example images of LFA strips captured using a cellphone at 2 min, 5 min, 10 min, 1 h, and 24 h after sample was applied. Fentanyl concentration of sample applied to the strip on the left: 0.1 ng/mL; right: 2 ng/mL.

FIG. 9 provides exemplary stability of LFA strips after storage. Two batches of strips were prepared on May 23, 2019, and Aug. 26, 2019, respectively. The batch prepared in May was stored in a sealed package at room temperature. Both batches were tested on Sep. 15, 2019 simultaneously. Fentanyl concentrations tested were 0.1 and 2 ng/mL, respectively.

FIG. 10 provides a simplified operational procedure for an example LFA strip. The Ab-AuNP conjugates (2 μL) were pre-aliquoted in the tubes. Different concentrations of fentanyl (0, 1, and 10 ng/mL; 200 μL) were added into each tube using an exact volume transfer pipet and mixed. Then, 50 μL mixture was applied using another exact volume transfer pipet to the LFA strips.

FIGS. 11A-11B provide clinical information and results for samples confirmed positive for fentanyl or norfentanyl in the LC-MS/MS method. Drug concentrations below the lower limits of quantitation of the LC-MS/MS method are indicated as ND. N/A indicates LC-MS/MS was not run on these samples or results were not available. Drug concentration unit is ng/mL.

FIG. 12 provides fentanyl and norfentanyl concentrations and result interpretations for quantifiable samples. Concentrations were quantitated using LC-MS/MS, and the interpretations were determined using each method's cutoffs. ND indicates analyte below lower limit of quantitation on the LC-MS/MS.

### DETAILED DESCRIPTION OF ILLUSTRATIVE EMBODIMENTS

The present disclosure can be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention.

Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable, and it should be understood that steps can be performed in any order.

It is to be appreciated that certain features of the invention which are, for clarity, described herein in the context of separate embodiments, can also be provided in combination in a single embodiment. Conversely, various features of the invention that are, for brevity, described in the context of a single embodiment, can also be provided separately or in any subcombination. All documents cited herein are incorporated herein in their entireties for any and all purposes.

Further, reference to values stated in ranges include each and every value within that range. In addition, the term “comprising” should be understood as having its standard, open-ended meaning, but also as encompassing “consisting” as well. For example, a device that comprises Part A and Part B can include parts in addition to Part A and Part B, but can also be formed only from Part A and Part B.

There is a long-felt need for rapid screening of analytes, e.g., opiods and other drugs of abuse. Existing screening techniques, however, are deficient in speed and/or sensitivity and can, in some cases, require operation by personnel trained in operation of medical diagnostics. Accordingly, there is a long-felt need for improved analyte screening devices and methods.

Fentanyl (a highly potent opioid originally synthesized for analgesia) is a non-limiting example of such analytes. For purposes of illustration, fentanyl is discussed below, though it should be understood that the following discussion of fentanyl is non-limiting and that the devices and techniques described and used in connection with fentanyl are illustrative only and do not limit the present disclosure. Accordingly, although certain examples and embodiments herein relate to fentanyl detection in samples taken from human patients, it should be understood that the present disclosure is not limited to fentanyl detection or to human samples. The disclosed technology can be applied to the detection of other analytes besides fentanyl, e.g., other drugs of abuse, biomolecules, pollutants, and the like. Similarly, the disclosed technology can be applied to samples collected from industrial processes, environmental samples, and the like, as the disclosed technology is not limited to the analysis of only human samples.

Fentanyl, a highly potent opioid originally synthesized for analgesia, has become a major contributing factor to the current opioid epidemic. The National Forensic Laboratory Information System reported dramatic increases in fentanyl-related encounters since 2014, especially in the Northeast and Midwest regions of the United States. As an example, fentanyl surpassed heroin as the leading drug involved in overdose deaths, accounting for 84% of opioid-related deaths in Philadelphia, resulting in a Department of Public Health notification to recommend implementing routine fentanyl testing in Emergency Departments (EDs). Similar trends have been observed in the European Union.

Although fentanyl and some analogs are used clinically as anesthetics and for pain management, most fentanyl-associated fatalities are caused by illicit fentanyl and analogs either alone or laced in other abused substances. In these situations, especially when the ingestion history includes only the presumed substance(s), which may have been adulterated with fentanyl, it is often difficult to recognize the presence of fentanyl and administer naloxone promptly based on clinical symptoms alone. Even in cases when fentanyl is an adulterant in other opioids such as heroin, and the clinical symptoms indicate the need for naloxone administration as an antidote, the recognition of fentanyl involvement is needed for the appropriate estimation of naloxone dose and frequency. Opioid type, amount and tolerance influence naloxone dose and frequency.

Due to the higher affinity of fentanyl for the μ-opioid receptors, larger and additional boluses or infusions of naloxone are needed to reverse respiratory depression caused by fentanyl, and a longer observation period preferably in the ED or hospital setting is usually warranted. Naloxone administration may precipitate unpleasant withdrawal symptoms, and has also led to adverse outcomes by increasing catecholamine release, leading to tachycardia, acute respiratory distress syndrome, and even death. Therefore, rapid identification of the causative drug as fentanyl is critical for prompt and appropriate administration of naloxone, and the right level of clinical care. From the public health perspective, rapid identification of fentanyl is also key to overdose surveillance, outbreak recognition and communication among healthcare professionals.

Fentanyl is metabolized to its main metabolite norfentanyl through oxidative N-dealkylation in the liver, with a short elimination half-life of 219 min. Norfentanyl was detected in all immediate postoperative urine specimens at concentrations of 5.4-11.5 ng/mL and still detectable up to 72 h at concentrations of 0.2 to 1.3 ng/mL in 7 adult female patients after receiving a single 50-100 μg intravenous fentanyl dose.

In a separate study, norfentanyl was detectable at concentrations of 1-18.3 ng/mL 1 h after 2,000-5,000 μg intravenous fentanyl dose. In overdose cases urine norfentanyl concentrations are likely higher than above. There is wide inter-individual variation in urine fentanyl concentrations present in samples from overdose or death cases. Urine fentanyl concentrations averaged 3.9 ng/mL in 112 adult intravenous abuse fatalities. Urine fentanyl concentrations ranged 5.0-93 ng/mL in 7 adults who died after self-administered intravenous injections of fentanyl. These data indicate the clinical need for screening methods to be able to detect fentanyl in urine samples at or below single-digit ng/mL concentrations, and norfentanyl around 10 ng/mL.

In contrast to the clinical demands, currently there is no rapid fentanyl screening method available to ED care providers, first responders, paramedics or laypersons, that can detect fentanyl at or below single-digit ng/mL concentrations and norfentanyl around 10 ng/mL, in the point-of-care settings. Gas chromatography-mass spectrometry or liquid chromatography tandem mass spectrometry (LC-MS/MS)-based methods are able to definitively quantify fentanyl as low as 1-2 ng/mL, but require too long a turn-around-time to be useful for onsite, rapid fentanyl screening.

Automated immunoassays are available from ARK Diagnostics, Immunalysis Corporation and Thermo Scientific. Neither of the latter two assays cross-reacts with norfentanyl, the major metabolite of fentanyl, while risperidone and 9-hydroxyrisperidone cross-react with the Thermo Scientific method. These assays have cutoffs at 1-2 ng/mL for urine fentanyl and can be adapted to automated immunoassay platforms, but are not suitable for point-of-care use. Lateral flow assays (LFAs) are available with cutoffs of 10-20 ng/mL for fentanyl and 100 ng/mL for norfentanyl, but these assays would yield false negative results in urines with fentanyl and norfentanyl concentrations below these cutoffs.

In this disclosure, provided is, inter alia, an LFA that was able to detect urine fentanyl at 1 ng/mL and/or norfentanyl at 10 ng/mL within 5-10 min.

Exemplary Investigation

It was decided to investigate whether rapid urine fentanyl screening be achieved with high clinical sensitivity and specificity at the point-of-care. To perform this investigation, a lateral flow strip detecting urine fentanyl ≥1 ng/mL and norfentanyl ≥10 ng/mL was developed.

In a group of consecutive 218 patients presenting to the Emergency Department and for whom urine drug screens were ordered, the strip test demonstrated clinical sensitivity and specificity (95% confidence interval (CI)) of 100% (75.8-100%) and 99.5% (97.3-99.9%). The positive and negative predictive values (95% CI) were 92.3% (66.7-98.6%) and 100% (98.2-100%) in this group with fentanyl prevalence of 5.5%. In the 2nd group of 7 patients with clinical suspicion of fentanyl overdose, the strip demonstrated 100% concordance with the liquid chromatography tandem mass spectrometry gold standard. Thus, the strip test achieved high clinical sensitivity and specificity for rapid fentanyl screening.

Rapid identification at the point of care of fentanyl as the causative agent for a drug overdose is critical. Urine fentanyl concentrations in overdose cases cover a wide range, with the low end at single-digit ng/mL. In contrast, no rapid fentanyl strip test with cutoff at or below single-digit ng/mL is presently available. Thus, the objective was to develop a rapid fentanyl strip test that can detect urine fentanyl at 1 ng/mL, and to evaluate the performance of the strip in urine samples from the Emergency Department (ED) of an academic center.

Design, setting and participants: A lateral flow strip was developed for rapid screening of fentanyl in 10 minutes. The analytical sensitivity and specificity of the strip was evaluated. Urine samples from two groups of ED patients were tested using the lateral flow strip and a liquid chromatography tandem mass spectrometry (LC-MS/MS) method for fentanyl, and results were compared. The first group is consisted of 218 consecutive ED patients with urine drug-of-abuse screen orders. The second group is consisted of 7 ED patients with clinically suspected fentanyl overdose.

Results: The strip detected both fentanyl (≥1 ng/mL) and its major metabolite norfentanyl (≥10 ng/mL). The strip demonstrated no cross-reactivity with amphetamine, cocaine, morphine, tetrahydrocannabinol, methadone, buprenorphine, naloxone and acetaminophen at 1000 ng/mL, but showed 0.03%, 0.4% and 0.05% cross-reactivity with carfentanyl, risperidone and 9-hydroxyrisperidone, respectively. In 218 consecutive ED patients, the prevalence of cases with fentanyl ≥1 ng/mL or norfentanyl ≥10 ng/mL was 5.5%. The clinical sensitivity and specificity (95% confidence interval (CI)) of the strip were 100% (75.8-100%) and 99.5% (97.3-99.9%), respectively. The positive and negative predictive values (95% CI) were 92.3% (66.7-98.6%) and 100% (98.2-100%), respectively. The concordance between the results from fentanyl strip and LC-MS/MS was 100% in the 7 suspected fentanyl overdose cases (5 positive, 2 negative).

Conclusions: The lateral flow fentanyl strip test detected fentanyl and norfentanyl with high clinical sensitivity and specificity in the ED patient population with rapid fentanyl screening needs.

Materials and Methods

Materials and Chemicals

Fentanyl-BSA (80-1409) and mouse monoclonal fentanyl antibody (10-2446) were purchased from Fitzgerald, Inc. (North Acton, Mass., USA). All drug standards, gold(III) chloride trihydrate (HAuCl4, 520918), Surine™ negative urine control (S-020-50ML), bovine serum albumin (BSA, A7906), Tween-20 (Molecular Biology Grade, P9416) were purchased from Sigma-Aldrich, Inc. (St. Louis, Mo., USA). Goat anti-mouse IgG (ABGAM-0500) was purchased from Arista Biologicals, Inc. (Allentown, Pa., USA). Potassium carbonate (S25480) was purchased from Thermo Fisher Scientific, Inc. (Rockford, Ill., USA).

Phosphate-buffered saline (PBS) tablets (T9181, pH 7.4) were purchased from Clontech Laboratories, Inc. (Mountain View, Calif., USA). Polyethylene glycol (PEG) 3350 was purchased from GoldBio, Inc. (St. Louis, Mo., USA). Sodium citrate (567446) was purchased from EMD Millipore Corp. (Billerica, Mass., USA). The nitrocellulose membrane (FF 80HP) and absorbent paper (GB003) were purchased from GE Healthcare Life Sciences (Pittsburgh, Pa., USA). The backing card was purchased from DCN Dx (Carlsbad, Calif., USA). β-glucuronidase (DR2100) was purchase from Campbell (Rockford, Ill., USA).

Clinical Subjects

The first cohort of subjects consisted of 218 consecutive patients presenting to the ED during January to February of 2019, for whom urine drugs-of-abuse screens were ordered. The second cohort consisted of 7 cases presenting to the ED with high clinical suspicions for fentanyl overdose. All urine samples were de-identified and frozen at −80° C. after clinical testing was completed, until time of retrospective analysis using the LFA and LC-MS/MS. The study was approved by the Institutional Review Board at the University of Pennsylvania under Waiver of Consent.

Preparation and Conjugation of Gold Nanoparticles

A citrate reduction method was used to prepare the gold nanoparticles (AuNPs). Briefly, 100 ml of 1 mM HAuCl4 solution was boiled under stirring. Ten ml of 38.8 mM sodium citrate was preheated and added to the boiling HAuCl4 solution. The solution was stirred for 15 min and cooled down to room temperature. Transmission electron microscopy (JEOL-F200 transmission electron microscope) and UV-visible spectroscopy (Infinite M Plex plate reader) were used to characterize the synthetic AuNPs.

The AuNPs were functionalized with anti-fentanyl antibodies via a modified physical adsorption method as previously described. Briefly, the pH of the AuNP solution was first adjusted to 9.0 using 100 mM potassium carbonate solution. Then, 40 μg of anti-fentanyl antibodies was mixed with 10 mL of AuNPs solution overnight at 4° C. A solution of 1% BSA was further used to block the unreacted sites on the surface of the AuNPs for 2 h at 4° C. Finally, the functionalized AuNPs were collected and purified via 3 times of centrifugation at 4000 g for 20 min each. The AuNPs were re-suspended in 2.5 mL of PBS, pH 7.4, containing 0.1% BSA, and stored at 4° C. for further use. UV-visible spectroscopy was used during the functionalization process to monitor the concentration and size of the AuNPs.

Lateral Flow Strip Preparation

To prepare the lateral flow test strip, fentanyl-BSA antigens (0.8 mg/ml) and goat anti-mouse IgG (0.8 mg/mL) were dispensed on the nitrocellulose membranes as the test and control line, respectively. The dispenser was composed of a GenieTouch™ Syringe Pump (Kent Scientific Corp., CT, USA) and a Mini 3D Printer (Monoprice, Inc., CA, USA). The reagents were dispensed at 60 uL/min for 10 s on a 10 cm-wide substrate. Then the coated membranes were dried at 37° C. overnight. Strips with widths of 5 mm each were produced using a paper-cutting machine and stored at room temperature in a sealed package with silica gel.

Analytical Sensitivity and Specificity Determination

Fifty microliters of synthetic urine containing antibody-AuNP (Ab-AuNP) conjugates and different concentrations of analytes was mixed together and applied to the strip. The qualitative test results were read after 10 min. To verify the limit of detection of the LFA, three independent experiments were conducted, in which different concentrations of fentanyl (0, 0.25, 0.5, 1, 5, and 10 ng/mL) were spiked into synthetic urine and tested. Common drugs of abuse, treatment drugs and previously known interfering drugs for other fentanyl immunoassays were spiked into synthetic urine and tested on the LFA for analytical specificity determination. To quantify the reactivities with norfentanyl, carfentanil, risperidone and 9-hydroxyrisperidone, a laboratory Gel Doc™ XR+ System from Bio-Rad Laboratories, Inc. (Hercules, Calif., USA) was used to scan the strips. The intensities of test and control lines were quantified using the Image Lab™ and Image J software, respectively.

Clinical Urine Sample Lateral Flow Strip and LC-MS/MS Analysis

For an example LFA, 49 μL of urine was mixed with 0.5 μL of fentanyl antibody-AuNP conjugates in 0.1 mM PBS buffer supplemented with 0.1% BSA, 0.1% Tween-20 and 0.2% PEG-3350, and applied to the strip. After 10 min, qualitative results were read. Both the control and test lines were visible in negative results, while only the control lines were visible in positive results. The performer of the LFA was blinded to the LC-MS/MS results.

The LC-MS/MS assay was a targeted method designed to detect and quantify forty-five common drugs of abuse, benzodiazepines and opiates. Analysis was performed on an ABSciex 3200 QTrap interfaced with a Shimadzu liquid chromatograph using isotopically-labeled internal standards.

Solvent A (0.1% formic acid in water) and solvent B (0.1% formic acid in methanol) were used to provide a gradient as follows (time/B %): initial/10%, 2 min/25%, 4.50 min/80%, 4.51 min/85%, 7.30 min/85% and 7.40 min/10%. Run time was 8.00 min. A Restek Analytical 5 μm Ultra Biphenyl 50×2.1 mm (P/N 9109552) column was used in this method.

Urine sample preparation included first hydrolysis using β-glucuronidase for 2 h at 55° C., then liquid-liquid extraction using 20% (v/v) methanol and centrifugation at 21000 g for 15 min. Twenty μL of extracted sample was injected, and column temperature was 40° C. The LC-MS/MS assay had a limit of quantitation of 1 ng/ml for fentanyl and 2 ng/mL for norfentanyl, and a cutoff of 2 ng/mL for fentanyl and 10 ng/mL for norfentanyl, respectively. The performer of the LC-MS/MS was blinded to the LFA results.

Statistical Analysis

Clinical sensitivity, specificity, positive and negative predictive values and their 95% confidence intervals were calculated according to the Clinical and Laboratory Standards Institute EP12-A2 guideline.

Results

Working Principle of Fentanyl Strip

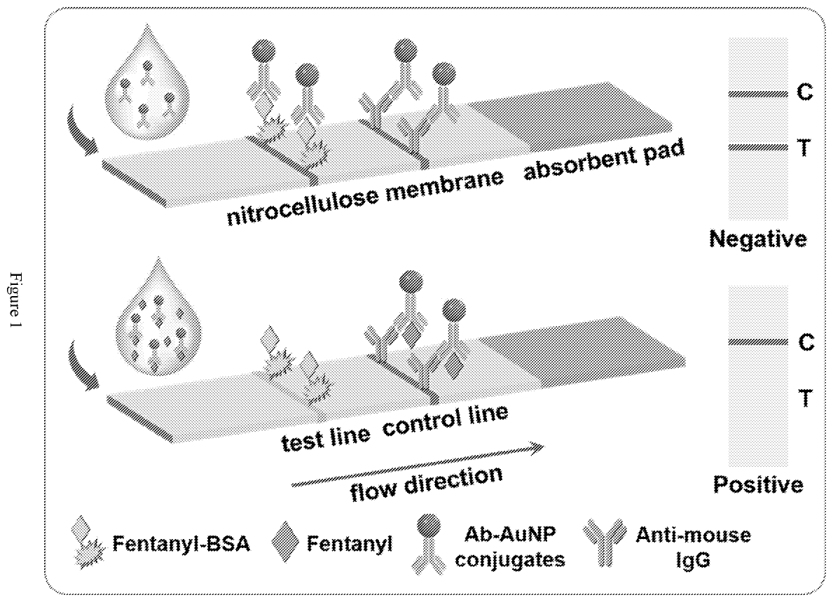

The fentanyl LFA was designed as a portable and low cost platform based on competitive lateral flow immunoassay. Its working principle is shown in FIG. 1. The pre-immobilized fentanyl-BSA on the test line competed with the target fentanyl molecule in the sample for binding to the antibody-AuNP conjugates. Antibody-AuNPs bound to the test and control lines were visible as red lines. As the fentanyl concentration in the sample increased, the color intensity of the test line decreased.

Antibody Gold Nanoparticle Conjugates

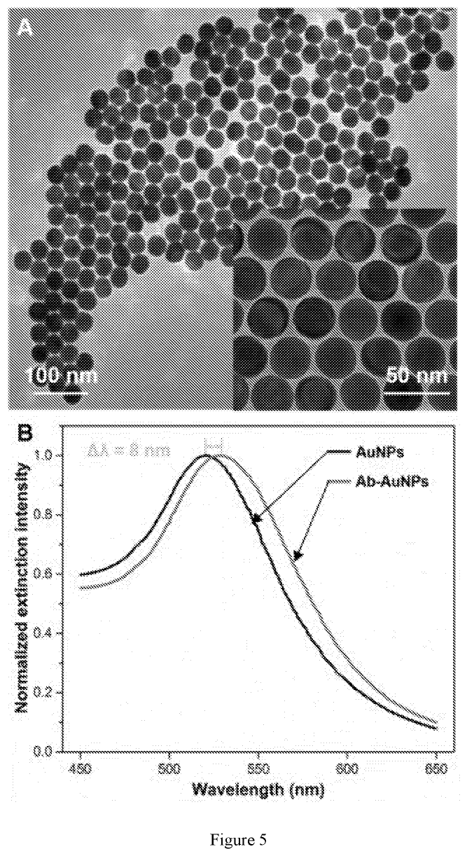

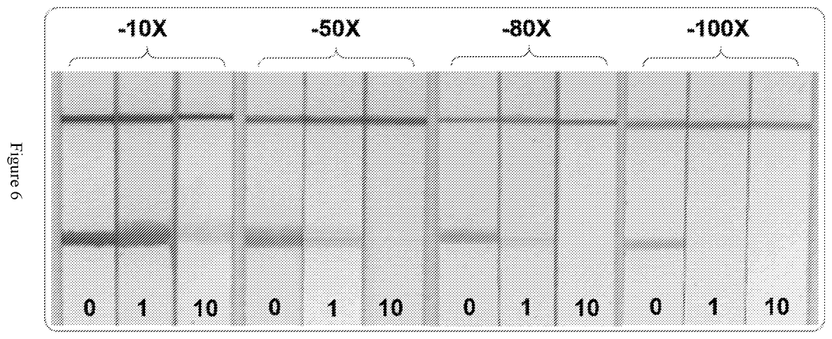

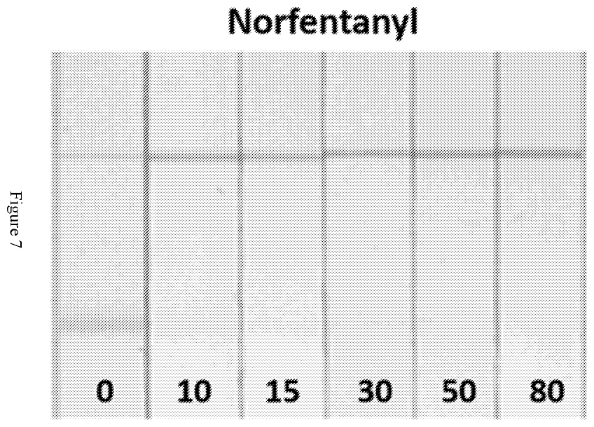

The transmission electron microscopy image of synthetic AuNPs (FIG. 5A) demonstrated that the AuNPs were homogenous in size and had a mean diameter of 30±2 nm. UV-visible spectroscopy was used during the functionalization process to characterize the conjugation of antibody and AuNPs (FIG. 5BB). AuNPs and Ab-AuNPs displayed an extinction maximum at 520 nm and 528 nm, respectively. These data supported that the size of the synthetic AuNPs was consistent and the anti-fentanyl antibodies successfully bound to the surface of AuNPs.

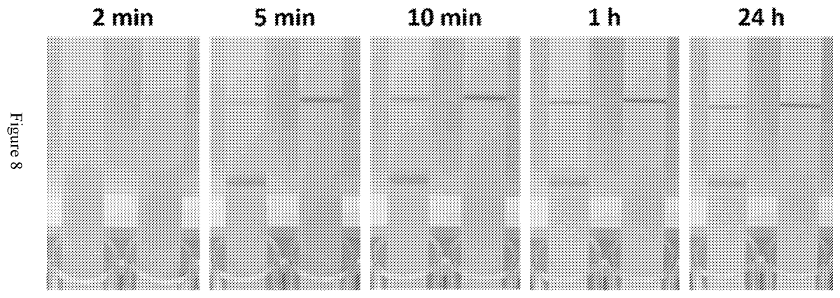

The concentration of Ab-AuNP conjugates was then investigated (FIG. 6). Different dilutions of Ab-AuNP conjugates were mixed with 0, 1 or 10 ng/mL fentanyl in synthetic urine. As the concentration of Ab-AuNPs decreased, the intensity of test and control lines also decreased, but the limit of detection was improved. A final concentration of 2.63×10⁹/mL Ab-AuNP conjugates (100×) was chosen for subsequent experiments.

Analytical Sensitivity and Specificity

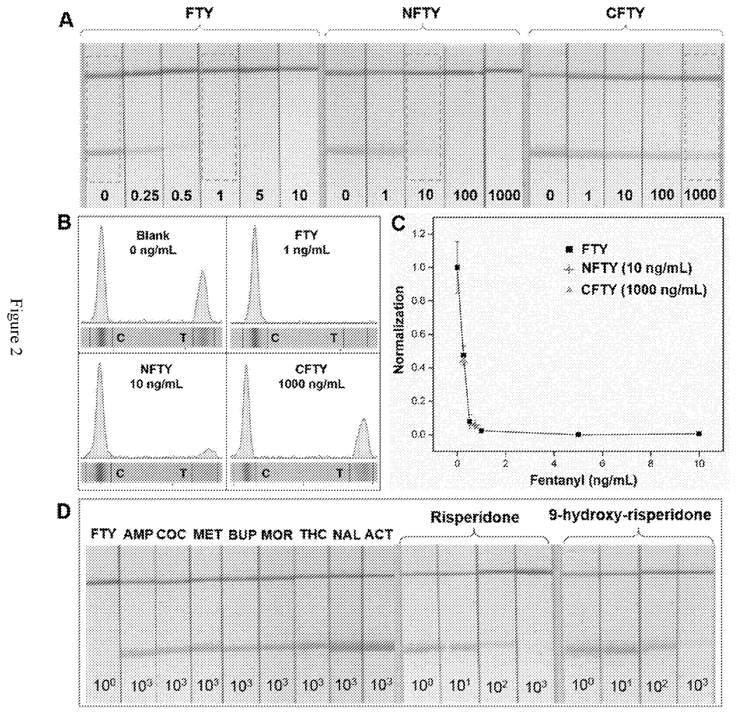

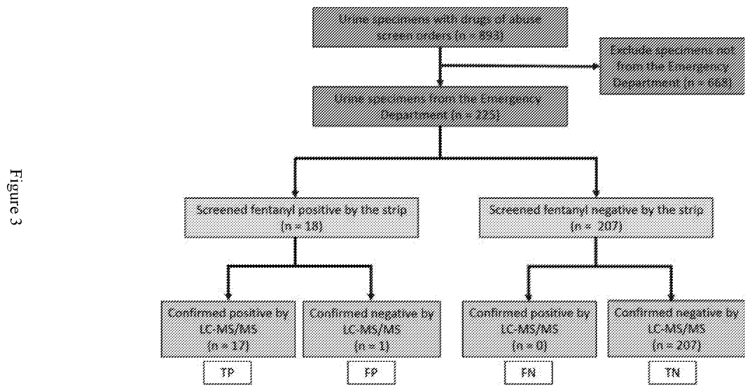

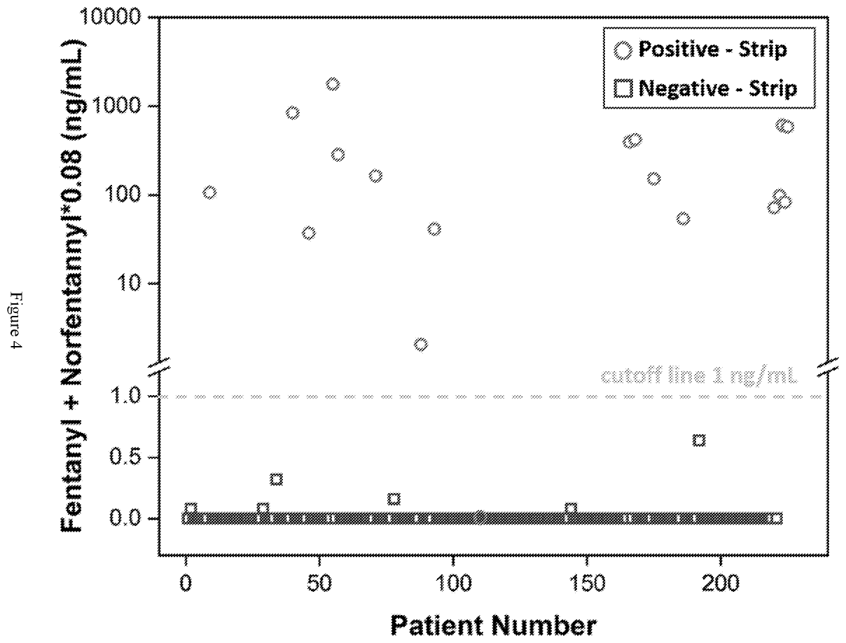

To characterize the limit of detection and analytical specificity of the fentanyl LFA, we tested synthetic urine samples spiked with different concentrations of fentanyl, norfentanyl and carfentanil (FIG. 2A). The signal intensity of test lines gradually decreased as the concentration of fentanyl increased from 0 to 1 ng/mL; when the concentrations were ≥1 ng/mL, the test lines were invisible (positive results). All the control lines were clearly visible. Similarly, norfentanyl ≥10 ng/mL led to positive results on the LFA. The results of additional concentrations of norfentanyl between 10 and 100 ng/mL are shown in FIG. 7. Result readout time window was characterized in FIG. 8, which showed that the result could be read as soon as 5 minutes, and was stable for at least 24 h. For standardizing purpose, results of all other LFA experiments were read at 10 min.

Negative results were observed for carfentanil up to 1000 ng/mL. To quantify norfentanyl and carfentanil reactivity, test and control lines in FIG. 2A dotted areas from triplicate experiments were scanned and quantified using densitometry (FIG. 2B). FIG. 2C shows the normalized intensity plot for fentanyl. From the plot, norfentanyl reactivity was calculated to be 8%, and carfentanil reactivity was 0.03%.

The analytical specificity of the fentanyl LFA was further characterized by testing several common drugs of abuse, treatment drugs and previously reported interfering drugs for other fentanyl assays. Of tested drugs, only risperidone (reactivity 0.4%) and its major metabolite 9-hydroxyrisperidone (reactivity 0.05%) cross-reacted in the fentanyl LFA (FIG. 2D).

The analytical precision of the fentanyl LFA was characterized and shown in Table 1, below. The lateral flow assay demonstrated precision from cutoff−100% to cutoff+100%.

TABLE 1
Precision of exemplary fentanyl LFA.

| Fentanyl | | | | Percent |
|---|---|---|---|---|
| Concentration | Relative % | # of | | Positive |
| (ng/mL) | Cutoff | Results | Results | Results % |
| 0 | −100 | 20 | 20 Negative | 0 |
| 0.5 | −50 | 20 | 20 Negative | 0 |
| 0.75 | −25 | 20 | 1 Positive; | 5 |
| | | | 19 Negative | |
| 1 | Cutoff | 20 | 11 Positive; | 55 |
| | | | 9 Negative | |
| 1.25 | +25 | 20 | 18 Positive; | 90 |
| | | | 2 Negative | |
| 1.5 | +50 | 20 | 20 Positive | 100 |
| 2 | +100 | 20 | 20 Positive | 100 |

Without being bound to any particular theory (and by reference to the non-limiting fentanyl example provided herein), the amount of fentanyl-BSA (i.e., the analyte conjugate) can be selected such that the amount is sufficient to capture at least the majority of the Ab-AuNP (i.e., detection complex) when the fentanyl (analyte) concentration in tested sample is just below 1 ng/mL (i.e., the selected cutoff value). The diameter of the nanoparticles can influence the amount of antibody conjugated on the nanoparticle surface and thus also influence the color intensity of the lines.

Clinical Validation of the Fentanyl Strip

To validate the performance of the fentanyl LFA, two cohorts of clinical urine samples were tested using both the fentanyl LFA and an LC-MS/MS method validated according to Clinical Laboratory Improvement Act standards. The first cohort of urine samples consisted of 218 consecutive urines from the ED with drugs-of-abuse screen orders. The STARD diagram is shown in FIG. 3. Patient characteristics are listed in Table 2, below.

TABLE 2
Demographic characteristics of subjects
in the consecutive ED patient cohort.

| All subjects | | n = 225 | |
|---|---|---|---|
| Gender | Male (%) | 113 | (50.2%) |
| | Female (%) | 112 | (49.8%) |
| Height (meters), mean (SD, range) | | 1.7 | (0.1, 1.5-2.1) |
| Weight (kilograms), mean (SD, range) | | 81.8 | (23.2, 46.7-206.4) |
| Age (years), mean (SD, range) | | 43 | (17, 18-88) |
| Race | Asian/Asian American (%) | 5 | (2.2%) |
| | Black/African American (%) | 145 | (64.4%) |
| | White/Caucasian (%) | 65 | (28.9%) |
| | Other (%) | 5 | (2.2%) |
| | Unknown (%) | 5 | (2.2%) |

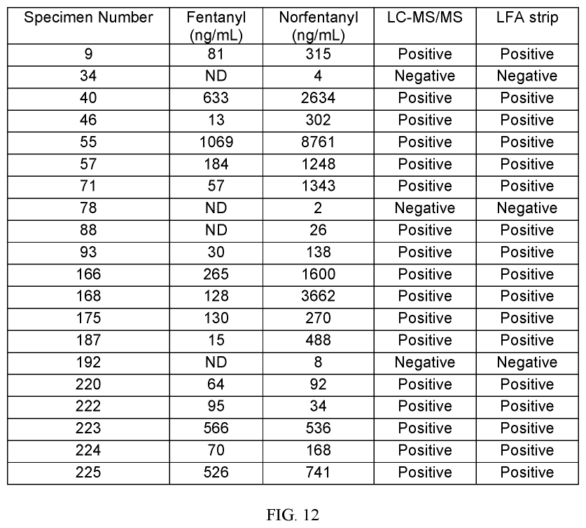

Individual patient results are plotted in FIG. 4. Data for all quantifiable samples is shown in FIG. 12.

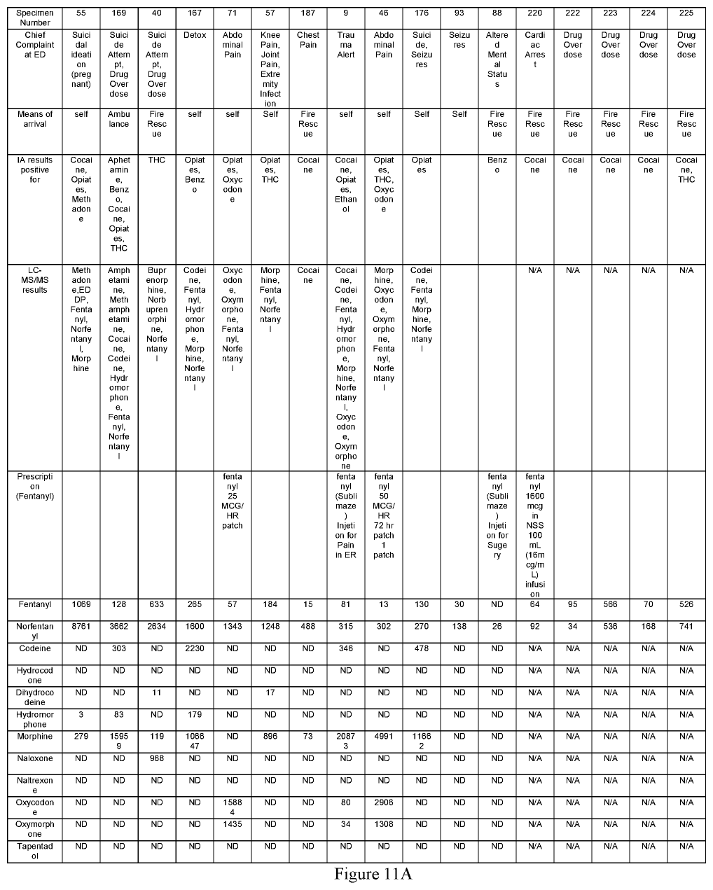

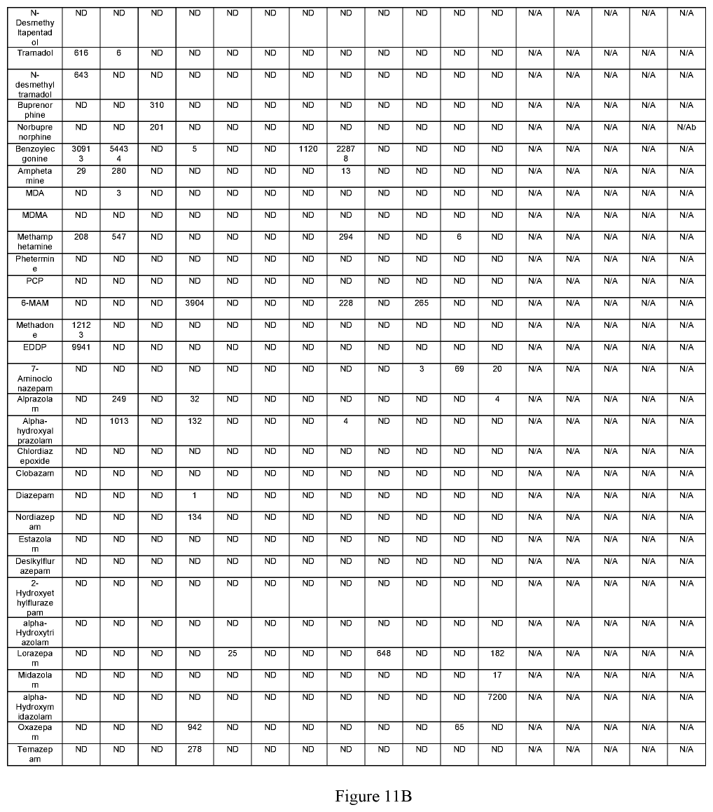

Using the LC-MS/MS as gold standard method, and 1 ng/mL fentanyl or 10 ng/mL norfentanyl as cutoffs, the clinical sensitivity of the fentanyl LFA was calculated to be 100% (95% confidence interval (CI) 75.8-100%), and the clinical specificity was 99.5% (95% CI 97.3-99.9%). The incidence of fentanyl/norfentanyl-positivity in this ED population was 5.5%. The positive and negative predictive values (95% CI) of the fentanyl LFA were 92.3% (66.7-98.6%) and 100% (98.2-100%) respectively. Clinical information of the 12 confirmed fentanyl-positive cases, including reasons for ED visit, drugs-of-abuse screening and LC-MS/MS results and prescriptions are listed in FIGS. 11A-11B. Morphine (positive rate 67%), codeine and benzoylecgonine (each 33%), methamphetamine and 6-monoacetylmorphine (each 25%), and oxycodone and oxymorphone (each 17%) were present most frequently in fentanyl-positive cases.

The second cohort of urine samples consisted of 7 cases in a clustered outbreak with suspected fentanyl contamination or adulteration into cocaine. All patients had a history of nonopioid substance-use disorder and no history of opioid use, and were brought by emergency medical services to the ED for suspected drug intoxication. All reported smoking “crack” cocaine but presented with opioid toxidrome.

Urine drug testing confirmed the presence of cocaine. Retrospective testing using the fentanyl LFA showed 100% concordance with LC-MS/MS results in this cohort (5 fentanyl positive, 2 fentanyl negative). The individual patient results are plotted in FIG. 4 as the last 7 data points. Two of the five fentanyl-positive cases required 2 mg, and another two required 4 mg naloxone to respond. The other fentanyl-positive case had multiple ED encounters during two days due to overdoses, requiring multiple doses of naloxone ranging 4-6 mg to respond, and eventually succumbed to cardiac arrest unresponsive to 8 mg naloxone.

Discussion

The fentanyl LFA described herein is the first point-of-care test that can detect fentanyl at a clinically relevant cutoff of 1 ng/mL fentanyl and 10 ng/mL for norfentanyl. Other commercial LFAs with cutoffs of 10-20 ng/mL for fentanyl, and 100 ng/mL for norfentanyl would yield more false negative results in screening.

Without being bound to any particular theory, the improved sensitivity may be attributed to two aspects. First is the choice of antibody, which cross reacts with both fentanyl and norfentanyl. Second is the optimization of assay reagents. The number of Ab-AuNPs conjugates was titrated to an amount such that the amount of fentanyl molecules present at the concentration of 1 ng/mL in the urine sample was sufficient to bind and capture all Ab-AuNPs, leaving no excess antibodies to be captured by the pre-immobilized fentanyl-BSA on the test line.

The LFA cross-reacted with risperidone and its major metabolite 9-hydroxyrisperidone at >100 and >1000 ng/mL respectively. Urine risperidone and 9-hydroxyrisperidone concentrations in overdose cases were reported to be 14.4-5600 and 17.8-2800 ng/mL respectively. Urine risperidone and 9-hydroxyrisperidone concentrations in geriatric patients taking therapeutic doses of risperidone were 0.32-22.3 and 1.87-24.8 ng/milk. Urines from patients taking risperidone may screen false positive for fentanyl using the LFA. Confirmation using mass spectrometry may be conducted when prescription or ingestion history includes risperidone.

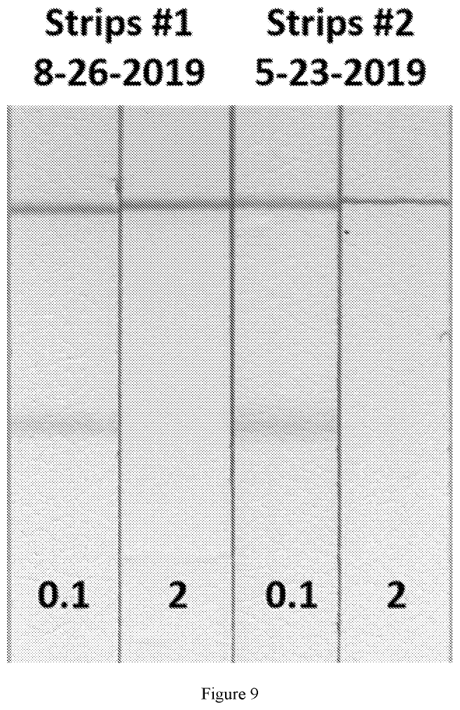

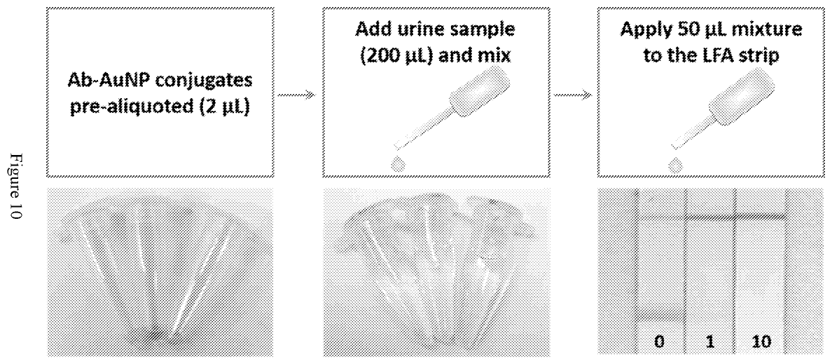

The fentanyl LFA demonstrated high clinical sensitivity and specificity in both cohorts of urine samples from the ED, showing that it is useful in screening and identifying patients with fentanyl overdose in an emergency. This allows prompt administration and optimal dosing of Naloxone, and direction of patients to the right level of clinical care. The results of fentanyl LFA strips remain valid for at least 24 h (FIG. 8). The strips can be stored in a sealed package at room temperature for at least three months and still maintain performance (FIG. 9).

In order to make the strip operation-friendly in emergency settings, fentanyl Ab-AuNP conjugates (e.g., 2 μL) can be pre-aliquoted into tubes as reagents supplied to the users. The user can then add (e.g., 200 μL) urine sample into the tube using a commercially-available exact volume transfer pipet (e.g., Universal Medical, FL, USA). After brief mixing by inverting the tube for a few times, the user can transfer some (e.g., 50 μL) of the mixture to the LFA strip, using another exact volume transfer pipet. This simplified procedure was tested in FIG. 10, which produced the same result as in FIG. 2A, demonstrating the testing procedure can be reduced to two simple volume transfers.

Of the 12 cases positive for fentanyl or norfentanyl in the first cohort of consecutive ED patients (FIGS. 11A-11B), 8 (67%) did not have a prescription history for fentanyl. Of the 4 positive cases with fentanyl prescription history, 2 were prescribed 25 and 50 μg/hr fentanyl patches, and the other 2 had history of fentanyl injections for pain control. The cases with fentanyl prescription history showed relatively low urine concentrations of fentanyl (0-81 ng/mL) and norfentanyl (26-1343 ng/mL), largely consistent with previous reports. In contrast, the cases with no fentanyl prescription history showed overall much higher urine concentrations of fentanyl (15-1069 ng/mL) and norfentanyl (138-8761 ng/mL). Other drugs of abuse most prevalent in fentanyl-positive cases were morphine, codeine and benzoylecgonine, methamphetamine and 6-monoacetylmorphine, oxycodone and oxymorphone. This indicates that fentanyl is most frequently co-ingested with other opioids (including heroin), cocaine and methamphetamine in this population, either knowingly or unknowingly.

One of the positive cases had no urine fentanyl and 26 ng/mL norfentanyl, which would be screened false negative using other LFAs. A few cases had low urine concentrations of norfentanyl (1-8 ng/mL) and no fentanyl detected on LC-MS/MS. As expected, these samples yielded negative results on the fentanyl LFA. These individuals may be near the end of fentanyl metabolism, who would test positive on the LFA earlier in the pharmacokinetic course. The LFA yielded 1 false positive result compared to LC-MS/MS. The source of false positivity in this case remains unclear, owing to the fact that all other drugs either positive in LC-MS/MS testing (tetrahydrocannabinol, oxymorphone) or on the patient's prescription list (buprenorphine, naloxone, acetaminophen and insulin) do not cross-react with the fentanyl LFA. It is possible that other drugs or metabolites not identified in the targeted LC-MS/MS method cross-reacted with the assay. A high-resolution screening LC-MS method with corresponding database can be helpful in identifying the cross-reacting sub stance.

One can note the prevalence of fentanyl and norfentanyl in the ED population during the study period was 5.5%, which was lower than THC (30.3%), benzoylecognine (11.9%) and morphine (8.2%), and the same as oxymorphone (5.5%) in the same population. Without being bound to any particular theory, the slightly lower fentanyl prevalence than what might be expected from national trend may be explained by the fact that the study was conducted in an academic medical center rather than a community setting or in the field, and the study duration was relatively short.

### Exemplary Embodiments

The following embodiments are exemplary only and do not serve to limit the scope of the present disclosure or the appended claims.

Embodiment 1. A screening device for screening a sample for an analyte, comprising: a pervious medium, the pervious medium comprising a test region and a control region; the test region comprising a conjugate of the analyte immobilized to the test region of the pervious medium, the control region comprising a control binding partner immobilized to the control region of the pervious medium, the control binding partner being complementary to a detection complex that comprises (i) a nanoparticle and (ii) a detection binding partner that is complementary to the analyte, and the test region (a) being visually perceptible following contact with a testing sample formed from at least the detection complex and a sample originally comprising the analyte at less than a cutoff concentration, and (b) being visually imperceptible following contact with a testing sample formed from at least the detection complex and a sample originally comprising the analyte at greater than a cutoff concentration.

It should be understood that a testing sample can be formed by adding detection complex directly to a sample (e.g., a urine sample) collected from a patient, which patient sample can be as-is, diluted, or otherwise modified. Thus, a testing sample can be formed by adding detection complex to a diluted sample (e.g., a urine sample) collected from a patient.

As an example, a testing sample can be formed from mixing detection complex with a patient sample that (before dilution or other processing) had therein 1 ng/mL fentanyl when originally collected from the patient. If the specified cutoff concentration is 2 ng/mL, then such a testing sample would be said to have the analyte (i.e., fentanyl) at less than the cutoff concentration. If the specified cutoff concentration is 0.5 ng/mL, then such a testing sample would be said to have the analyte at greater than the cutoff concentration. Thus, the term “cutoff concentration” refers to the concentration of the analyte in the original sample that is collected for analysis, and above which concentration the test result changes from absent (negative) to present (positive).

Pervious media can include, e.g., cellulose, paper, and other porous or fibrous materials. Nitrocellulose membranes are considered especially suitable, but other pervious media can be used. The analyte can be conjugated to, e.g., BSA. Other blockers besides BSA can be used.

A test region can be of virtually any shape, e.g., a stripe or a square. Likewise, a control region can also be of virtually any shape. Without being bound to any particular theory of operation, the control region can be configured to detect the presence of detection complex—without analyte—in a sample that is applied to the control region.

By reference to non-limiting FIG. 1, goat anti-mouse IgG is immobilized on the control region, which IgG can capture the Ab-AuNP detection complex, whether or not the detection complex binds to the analyte (fentanyl, in the case of FIG. 1). If Ab-AuNP detection complex binds to fentanyl first, then the detection complex cannot bind to the analyte conjugate fentanyl-BSA shown in FIG. 1. Thus, there would be (again, by reference to FIG. 1) a visible control line in all situations (i.e., whether a negative or a positive sample). The control line can indicate that there is detection complex present in the reaction and that can be captured by the control binding partner (IgG, by reference to FIG. 1). For example, when the device has been stored for a long time, the red control line is used to indicate whether the device still works properly. Without being bound to any particular theory, if there is no red control line in the testing, then the test may warrant reevaluation, even if there is a test line on the test region.

A control binding partner can be a species that binds to the detection complex. As an example (see FIG. 1), goat anti-mouse IgG can be used as a control binding partner when the detection complex includes a mouse monoclonal fentanyl antibody. As another example, the control binding partner can be another antibody that is itself complementary to an antibody of the detection complex.

A detection complex can include a nanoparticle (described elsewhere herein) and a detection binding partner. Suitable detection binding partners include, e.g., antibodies, such as mouse monoclonal antibodies. Other antibodies are also suitable, e.g., polyclonal antibodies.

The visual perceptibility of the test region can refer to an ordinary individual being able to perceive an indicium (e.g., a color stripe) of the test region. As shown in the attached figures, visual perception can be based upon a visible color stripe. By reference to non-limiting FIG. 6, a color stripe is visible at the test strip corresponding to 0 ng/mL fentanyl at 100× dilution of detection complex and the color strip is not visible in the adjacent test strip corresponding to 1 ng/mL fentanyl at 100× dilution of detection complex.

In some embodiments, one can use an imaging device (e.g., a smartphone, a microscope, a camera, and the like) to obtain images of the test region and/or the control region.

Embodiment 2. The device of Embodiment 1, wherein the cutoff concentration of the analyte (i.e., the concentration of the analyte in an original sample of interest before that sample is combined with the detection complex) is from about 1 ng/mL to about 10 ng/mL, e.g., from 1 to about 10 ng/mL, from 1.5 to about 9.5 ng/mL, from 2.0 to about 9.0 ng/mL, from 2.5 to about 8.5 ng/mL, from 3.0 to about 8.0 ng/mL, from about 3.5 to about 7.5 ng/mL, from about 4.0 to about 7.0 ng/mL, from about 4.5 to about 6.5 ng/mL, from about 5 to about 6 ng/mL. Cutoff concentrations of 1 ng/mL, 1.1 ng/mL, 1.2 ng/mL, 1.3 ng/mL, 1.4 ng/mL, 1.5 ng/mL, 1.6 ng/mL, 1.7 ng/mL, 1.8 ng/mL, 1.9 ng/mL, 2.0 ng/mL, 2.1 ng/mL, 2.2 ng/mL, 2.3 ng/mL, 2.4 ng/mL, or 2.5 ng/mL (or any of the foregoing values or any range within the foregoing values) are considered suitable. Other cutoff concentrations can be used, as a user can set a cutoff concentration based on clinical or regulatory relevance.

Embodiment 3. The device of Embodiment 2, wherein the cutoff concentration of the analyte is about 1 ng/mL.

Embodiment 4. The device of Embodiment 1, wherein the nanoparticle of the detection complex has a diameter of from about 5 nm to about 100 nm. Nanoparticles having a diameter of from about 5 to about 100 nm, or from about 10 to about 90 nm, or from about 20 to about 80 nm, or from about 30 to about 70 nm, or from about 40 to about 60 nm, or even about 50 nm are all considered suitable. Nanoparticles having a diameter of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nm (or any of the foregoing values or any range within the foregoing values) are considered suitable.

Embodiment 5. The device of Embodiment 1, wherein the nanoparticle of the detection complex has a diameter of about 30 nm.

Embodiment 6. The device of any one of Embodiments 1-5, wherein the nanoparticle of the detection complex comprises a metal. A variety of metals can be used, e.g., silver, gold, iron, titanium,

Embodiment 7. The device of Embodiment 5, wherein the metal is gold.

Embodiment 8. The device of any one of Embodiments 1-7, wherein the detection complex is present in the sample at from about 2.5×10⁹/mL to about 2.8×10¹¹/mL. The detection complex can also be present in the sample at other concentrations, e.g., from 1×10⁷/mL to about 1×10^{13/}mL, from 1×10⁸/mL to about 1×10¹²/mL, from 1×10⁷/mL to about 1×10¹³/mL, from 1×10⁹/mL to about 1×10¹⁰/mL.

Embodiment 9. The screening device of any one of Embodiments 1-8, wherein the analyte of interest comprises an opioid.

Embodiment 10. The screening device of Embodiment 9, wherein the opioid comprises fentanyl.

Embodiment 11. The screening device of any one of Embodiments 1-8, wherein the analyte of interest comprises fentanyl, norfentanyl, codeine, hydrocodone, dihydrocodeine, hydromorphone, morphine, naloxone, naltrexone, oxycodone, oxymorphone, tapentadol, n-desmethyltapentadol, tramadol, N-desmethyltramadol, buprenorphine, norbuprenorphine, benzoylecgonine, amphetamine, MDA, MDMA, methamphetamine, phetermine, PCP, 6-MAM, methadone, EDDP, 7-aminoclonazepam, alprazolam, alpha-hydroxyalprazolam, chlordiazepoxide, clobazam, diazepam, nordiazepam, estazolam, deslkylflurazepam, 2-hydroxyethylflurazepam, alpha-hydroxytriazolam, lorazepam, midazolam, alpha-hydroxymidazolam, oxazepam, carfentanil, or temazepam. The analyte can also be a metabolite of any of the foregoing.

Embodiment 12. The screening device of any one of Embodiments 1-11, wherein the detection binding partner comprises an antibody. As one of ordinary skill will appreciate, one can purchase an antibody complementary to the analyte of interest, e.g., mouse monoclonal fentanyl antibody from Fitzgerald, Inc. (North Acton, Mass., USA), and polyclonal anti-fentanyl antibodies.

Embodiment 13. A screening device for screening a sample, comprising: a pervious medium, the pervious medium comprising a test region and a control region; the test region comprising a conjugate of an analyte immobilized to the pervious medium, the control region comprising a control binding partner immobilized to the pervious medium, the control binding partner being complementary to a detection complex that comprises (i) a nanoparticle and (ii) a detection partner complementary to the analyte of interest, and wherein the test region comprises a visually perceptible level of the detection complex following contact with a sample that comprises the detection complex and less than about 1 ng/mL of the analyte.

Embodiment 14. A screening method, comprising: contacting a sample with an amount of a detection complex, the detection complex comprising a (i) nanoparticle and (ii) a detection partner complementary to an analyte, the contacting giving rise to a treated sample; introducing the treated sample to a pervious medium, the pervious medium comprising a test region and a control region, the test region comprising a conjugate of the analyte immobilized to the test region of the pervious medium, the control region comprising a control binding partner immobilized to the control region of the pervious medium, wherein the amount of the detection complex is selected such that the test region is (a) visually perceptible following contact with a testing sample formed from at least the detection complex and a sample originally comprising the analyte at less than a cutoff concentration, and (b) visually imperceptible following contact with a testing sample formed from at least the detection complex and a sample originally comprising the analyte at greater than a cutoff concentration.

Suitable detection complexes, nanoparticles, detection partners, analytes, pervious media, test regions, control regions, control binding partners, conjugates of analytes, samples, and cutoff concentrations are described elsewhere herein.

Embodiment 15. The method of Embodiment 14, wherein the detection complex is present in the treated sample at from about 2.5×10⁹/mL to about 2.8×10¹¹/mL.

Embodiment 16. The method of Embodiment 15, wherein the detection complex is present in the treated sample at about 2.5×10⁹/mL.

Embodiment 17. The method of any one of Embodiments 14-16, wherein the analyte comprises any one (or more) of fentanyl, norfentanyl, codeine, hydrocodone, dihydrocodeine, hydromorphone, morphine, naloxone, naltrexone, oxycodone, oxymorphone, tapentadol, n-desmethyltapentadol, tramadol, N-desmethyltramadol, buprenorphine, norbuprenorphine, benzoylecgonine, amphetamine, MDA, MDMA, methamphetamine, phetermine, PCP, 6-MAM, methadone, EDDP, 7-aminoclonazepam, alprazolam, alpha-hydroxyalprazolam, chlordiazepoxide, clobazam, diazepam, nordiazepam, estazolam, deslkylflurazepam, 2-hydroxyethylflurazepam, alpha-hydroxytriazolam, lorazepam, midazolam, alpha-hydroxymidazolam, oxazepam, canfentanil, or temazepam. The analyte can also comprise a metabolite of the foregoing.

Embodiment 18. The method of any one of Embodiments 14-17, wherein the sample comprises a body fluid sample, a tissue sample, any combination thereof, or any extractant (or combination thereof) of such samples. Exemplary body fluid samples include, e.g., saliva, urine, blood, mucus, semen, vaginal fluid, lymph fluid, joint fluid, and the like. Tissue samples include muscle, skin, hair, nails, and the like.

Embodiment 19. The method of any one of Embodiments 14-18, wherein the detection partner comprises an antibody.

Embodiment 20. The method of any one of Embodiments 14-19, wherein the nanoparticle has a diameter of from about 5 nm to about 100 nm.

Embodiment 21. The method of any one of Embodiments 14-20, further comprising interrogating the test region for visual perceptibility. Interrogation can be performed in a manual fashion, but can also be performed in an automated fashion as well. Interrogation can be performed by a smartphone or other mobile device.

A user can also (manually or automatically) compare one or more attributes of a test region (e.g., darkness, color, color intensity) to a standard for that attribute. As an example a user can compare the intensity of a color of a test region against one or more “standards” that are stored in a memory or that are present on a standards card. In this way, a user can determine which standard has the closest “match” to the test region.

For example, if the color at a given test region most closely matches the color of a standard that corresponds to a level of a particular drug of abuse of 5 ng/mL, a user can then estimate that the level of the drug of abuse in the sample in question is about 5 ng/mL.

Embodiment 22. A kit, comprising: (i) a screening device for screening a sample for an analyte, comprising: a pervious medium, the pervious medium comprising a test region and a control region; the test region comprising a conjugate of the analyte immobilized to the test region of the pervious medium, the control region comprising a control binding partner immobilized to the control region of the pervious medium, the control binding partner being complementary to a detection complex that comprises (1) a nanoparticle and (2) a detection binding partner that is complementary to the analyte, and the test region (a) being visually perceptible following contact with a testing sample formed from at least the detection complex and a sample originally comprising the analyte at less than a cutoff concentration, and (b) being visually imperceptible following contact with a testing sample formed from at least the detection complex and a sample originally comprising the analyte at greater than a cutoff concentration; and (ii) a supply of the detection complex.

The supply of the detection complex can comprise one, two, three, or more different detection complexes. In this way, a user can contact the supply of detection complex to a sample in preparation for detecting multiple analytes in the sample. The supply of the detection complex can be stored with the screening device, but this is not a requirement, as they can be stored separately.

As one example, a supply of detection complex can include detection complexes that are specific to analyte A and also include detection complexes that are specific to analyte B. In this way, a user can then in turn screen a sample for the presence of both analyte A and analyte B. The kit can include a pervious medium that includes test and control regions configured to test (and control) for analyte A and analyte B, thus allowing for multiplexed screening. The pervious medium can include lanes or regions that are separate or even in fluidic isolation from one another. Alternatively, a pervious medium can be configured to receive a sample and then the sample is directed (e.g., via a manifold, via capillary or wicking action to two or more regions, each of which regions is configured to screen for a different analyte.

Embodiment 23. The kit of Embodiment 22, further comprising a diluent configured for addition to the supply of the detection complex. A diluent can be, e.g., water, a buffer, and the like.

Embodiment 24. The kit of any one of Embodiments 22-23, wherein the supply of the detection complex comprises the detection complex at a concentration selected such that the test region is (a) visually perceptible following contact with a sample that comprises the detection complex and the analyte less than a cutoff concentration, and (b) visually imperceptible following contact with a sample that comprises the detection complex and the analyte greater than the cutoff concentration.

Embodiment 25. The kit of any one of Embodiments 22-24, wherein the kit comprises a plurality of test regions, each of the test regions comprising a conjugate of one A of n different analytes A₁-Aₙ, and wherein the kit comprises a plurality of supplies of detection complexes, each of the different complexes comprising a different detection binding partner that is complementary to a different one A of n different analytes A₁-Aₙ.

Embodiment 26. The kit of any one of Embodiments 22-25, wherein the cutoff concentration of the analyte is from, e.g., about 0.1 ng/mL to about 10,000 ng/mL or even to about 50,000 ng/mL. For example, a cutoff concentration can be, e.g., from about 0.2 ng/mL to about 20,000 ng/mL, from about 0.3 ng/mL to about 10,000 ng/mL, from about 0.5 ng/mL to about 100 ng/mL, from about 1 ng/mL to about 100 ng/mL, from about 2 ng/mL to about 50 ng/mL, from about 5 ng/mL to about 25 ng/mL, from about 0.1 to about 50 ng/mL, from about 0.5 to about 20 ng/mL, from about 0.5 to about 10 ng/mL, from about 1 to about 10 ng/mL, and any and all intermediate values and subranges. It should be understood that the foregoing cutoff concentrations and ranges are illustrative only and do not limit the scope of the disclosed technology.

Additional information can be found in “Development and Clinical Validation of a Sensitive Lateral Flow Assay for Rapid Urine Fentanyl Screening in the Emergency Department,” Li et al., Clinical Chemistry 66:2, 324-332 (2020), the entirety of which is incorporated herein by reference for any and all purposes.

### REFERENCES

The following references are listed for convenience only and are incorporated herein in their entireties for any and all purposes.
Jannetto P J, Helander A, Garg U, Janis G C, Goldberger B, Ketha H. The Fentanyl Epidemic and Evolution of Fentanyl Analogs in the United States and the European Union. Clin Chem. 2019; 65(2):242-253.
Health PDoP. Recommendation to Implement Routine Fentanyl Testing in Hospital Emergency Departments. 2018; https://hip.phila.gov/Portals/default/HIP/HealthAlerts/2018/PDPH-HAN_Notification_5_RapidFentanylTesting_10302018.pdf. Accessed Mar. 19, 2019, 2019.
Li K, Armenian P, Mason J, Grock A. Narcan or Nar-can't: Tips and Tricks to Safely Reversing Opioid Toxicity. Ann Emerg Med. 2018; 72(1):9-11.
Armenian P, Vo K T, Barr-Walker J, Lynch K L. Fentanyl, fentanyl analogs and novel synthetic opioids: A comprehensive review. Neuropharmacology. 2018; 134(Pt A):121-132.
Henderson G L. Fentanyl-related deaths: demographics, circumstances, and toxicology of 112 cases. J Forensic Sci. 1991; 36(2):422-433.
Ferner R. Disposition of toxic drugs and chemicals in man 11th edition. Clin Toxicol (Phila). 2018; 56(3):234.
Wang B T, Colby J M, Wu A H, Lynch K L. Cross-reactivity of acetylfentanyl and risperidone with a fentanyl immunoassay. J Anal Toxicol. 2014; 38(9):672-675.
Helander A, Stojanovic K, Villen T, Beck O. Detectability of fentanyl and designer fentanyls in urine by 3 commercial fentanyl immunoassays. Drug Test Anal. 2018.
Abbas A, Fei M, Tian L, Singamaneni S. Trapping proteins within gold nanoparticle assemblies: dynamically tunable hot-spots for nanobiosensing. Plasmonics. 2013; 8(2):537-544.
Liu J, Hu X, Cao F, Zhang Y, Lu J, Zeng L. A lateral flow strip based on gold nanoparticles to detect 6-monoacetylmorphine in oral fluid. Royal Society open science. 2018; 5(6):180288.
Liu X, Atwater M, Wang J, Huo Q. Extinction coefficient of gold nanoparticles with different sizes and different capping ligands. Colloids and Surfaces B: Biointerfaces. 2007; 58(1):3-7.
Tripathi K, Driskell J D. Quantifying Bound and Active Antibodies Conjugated to Gold Nanoparticles: A Comprehensive and Robust Approach To Evaluate Immobilization Chemistry. ACS Omega. 2018; 3(7):8253-8259.
Quesada-Gonzalez D, Merkoci A. Nanoparticle-based lateral flow biosensors. Biosensors and Bioelectronics. 2015; 73:47-63.
Khatri U G, Viner K, Perrone J. Lethal Fentanyl and Cocaine Intoxication. N Engl J Med. 2018; 379(18):1782.
Serebrennikova K, Samsonova J, Osipov A. Hierarchical nanogold labels to improve the sensitivity of lateral flow immunoassay. Nano-micro letters. 2018; 10(2):24.
Aveyard J, Mehrabi M, Cossins A, Braven H, Wilson R. One step visual detection of PCR products with gold nanoparticles and a nucleic acid lateral flow (NALF) device. Chemical Communications. 2007(41):4251-4253.
Safenkova I, Zherdev A, Dzantiev B. Factors influencing the detection limit of the lateral-flow sandwich immunoassay: a case study with potato virus X. Analytical and bioanalytical chemistry. 2012; 403(6):1595-1605.
Lee H S, Tan C H, Au L S, Khoo Y M. Serum and urine risperidone concentrations in an acute overdose. Journal of clinical psychopharmacology. 1997; 17(4):325-326.
Springfield A C, Bodiford E. An overdose of risperidone. Journal of analytical toxicology. 1996; 20(3):202-203.
Maxwell R A, Sweet R A, Mulsant B H, et al. Risperidone and 9-hydroxyrisperidone concentrations are not dependent on age or creatinine clearance among elderly subjects. Journal of geriatric psychiatry and neurology. 2002; 15(2):77-81.
Boyer E W. Management of opioid analgesic overdose. New England Journal of Medicine. 2012; 367(2):146-155.
Armenian P, Vo K T, Barr-Walker J, Lynch K L. Fentanyl, fentanyl analogs and novel synthetic opioids: a comprehensive review. Neuropharmacology. 2018; 134:121-132.
Cummings O T, Enders J R, McIntire G L, Backer R, Poklis A. Fentanyl-Norfentanyl Concentrations During Transdermal Patch Application: LC-MS-MS Urine Analysis. J Anal Toxicol. 2016; 40(8):595-600.
Poklis A, Backer R. Urine concentrations of fentanyl and norfentanyl during application of Duragesic transdermal patches. Journal of analytical toxicology. 2004; 28(6):422-425.

1. A screening device for screening a sample for an analyte, comprising:
a pervious medium, the pervious medium comprising a test region and a control region;
the test region comprising a conjugate of the analyte immobilized to the test region of the pervious medium,
the control region comprising a control binding partner immobilized to the control region of the pervious medium,
the control binding partner being complementary to a detection complex that comprises (i) a nanoparticle and (ii) a detection binding partner that is complementary to the analyte, and
the test (a) being visually perceptible following contact with a testing sample formed from at least the detection complex and a sample originally comprising the analyte at less than a cutoff concentration, and (b) being visually imperceptible following contact with a testing sample formed from at least the detection complex and a sample originally comprising the analyte at greater than a cutoff concentration.

2. The device of claim 1, wherein the cutoff concentration of the analyte is from about 0.5 ng/mL to about 200 ng/mL.

3. The device of claim 2, wherein the cutoff concentration of the analyte is about 1 ng/mL.

4. The device of claim 1, wherein the nanoparticle of the detection complex has a diameter of from about 5 nm to about 100 nm.

5. (canceled)

6. The device of claim 1, wherein the nanoparticle of the detection complex comprises a metal.

7. The device of claim 6, wherein the metal is gold.

8. The device of claim 1, wherein the detection complex is present in the sample at from about 2.5×10⁹/mL to about 2.8×10¹¹/mL.

9. The screening device of claim 1, wherein the analyte of interest comprises an opioid.

10. The screening device of claim 9, wherein the opioid comprises fentanyl.

11. The screening device of claim 1, wherein the analyte of interest comprises fentanyl, norfentanyl, codeine, hydrocodone, dihydrocodeine, hydromorphone, morphine, naloxone, naltrexone, oxycodone, oxymorphone, tapentadol, n-desmethyltapentadol, tramadol, N-desmethyltramadol, buprenorphine, norbuprenorphine, benzoylecgonine, amphetamine, MDA, MDMA, methamphetamine, phetermine, PCP, 6-MAM, methadone, EDDP, 7-aminoclonazepam, alprazolam, alpha-hydroxyalprazolam, chlordiazepoxide, clobazam, diazepam, nordiazepam, estazolam, deslkylflurazepam, 2-hydroxyethylflurazepam, alpha-hydroxytriazolam, lorazepam, midazolam, alpha-hydroxymidazolam, oxazepam, or temazepam.

12. The screening device of claim 1, wherein the detection binding partner comprises an antibody.

13. A screening device for screening a sample, comprising:
a pervious medium, the pervious medium comprising a test region and optionally a control region;
the test region comprising a conjugate of an analyte immobilized to the pervious medium,
the control region comprising a control binding partner immobilized to the pervious medium,
the control binding partner being complementary to a detection complex that comprises (i) a nanoparticle and (ii) a detection partner complementary to the analyte of interest, and
wherein the test region comprises a visually perceptible level of the detection complex following contact with a sample formed from at least the detection complex and a sample originally comprising less than about 1 ng/mL of the analyte.

14. A screening method, comprising:
contacting a sample with an amount of a detection complex, the detection complex comprising a (i) nanoparticle and (ii) a detection partner complementary to an analyte, the contacting giving rise to a treated sample;
introducing the treated sample to a pervious medium, the pervious medium comprising a test region and optionally a control region,
the test region comprising a conjugate of the analyte immobilized to the test region of the pervious medium,
the control region comprising a control binding partner immobilized to the control region of the pervious medium,
wherein the amount of the detection complex is selected such that the test region is (a) visually perceptible following contact with a testing sample formed from at least the detection complex and a sample originally comprising the analyte at less than a cutoff concentration, and (b) visually imperceptible following contact with a testing sample formed from at least the detection complex and a sample originally comprising the analyte at greater than a cutoff concentration.

15. The method of claim 14, wherein the detection complex is present in the treated sample at from about 2.5×10⁹/mL to about 2.8×10¹¹/mL.

16. (canceled)

17. The method of claim 14, wherein the analyte comprises fentanyl, norfentanyl, codeine, hydrocodone, dihydrocodeine, hydromorphone, morphine, naloxone, naltrexone, oxycodone, oxymorphone, tapentadol, n-desmethyltapentadol, tramadol, N-desmethyltramadol, buprenorphine, norbuprenorphine, benzoylecgonine, amphetamine, MDA, MDMA, methamphetamine, phetermine, PCP, 6-MAM, methadone, EDDP, 7-aminoclonazepam, alprazolam, alpha-hydroxyalprazolam, chlordiazepoxide, clobazam, diazepam, nordiazepam, estazolam, deslkylflurazepam, 2-hydroxyethylflurazepam, alpha-hydroxytriazolam, lorazepam, midazolam, alpha-hydroxymidazolam, oxazepam, or temazepam.

18. The method of claim 14, wherein the sample comprises a body fluid sample, a tissue sample, or any combination thereof, or any extractant of such samples.

19. The method of claim 14, wherein the detection partner comprises an antibody.

20. (canceled)

21. The method of claim 14, further comprising interrogating the test region for visual perceptibility.

22. A kit, comprising:
(i) a screening device for screening a sample for an analyte, comprising:
a pervious medium, the pervious medium comprising a test region and optionally a control region;
the test region comprising a conjugate of the analyte immobilized to the test region of the pervious medium,
the control region comprising a control binding partner immobilized to the control region of the pervious medium,
the control binding partner being complementary to a detection complex that comprises (1) a nanoparticle and (2) a detection binding partner that is complementary to the analyte, and
the test region (a) being visually perceptible following contact with a testing sample formed from at least the detection complex and a sample originally comprising the analyte at less than a cutoff concentration, and (b) being visually imperceptible following contact with a testing sample formed from at least the detection complex and a sample originally comprising the analyte at greater than a cutoff concentration; and
(ii) a supply of the detection complex.

23. The kit of claim 22, further comprising a diluent configured for addition to the supply of the detection complex.

24. The kit of claim 22, wherein the supply of the detection complex comprises the detection complex at a concentration selected such that the test region is (a) visually perceptible following contact with a sample that comprises the detection complex and the analyte less than a cutoff concentration, and (b) visually imperceptible following contact with a sample that comprises the detection complex and the analyte greater than the cutoff concentration.

25. The kit of claim 22, wherein the kit comprises a plurality of test regions, each of the test regions comprising a conjugate of one A of n different analytes A₁-Aₙ, and wherein the kit comprises a plurality of supplies of detection complexes, each of the different complexes comprising a different detection binding partner that is complementary to a different one A of n different analytes A₁-Aₙ.

26. The kit of claim 22, wherein the cutoff concentration of the analyte is from about 0.5 ng/mL to about 500 ng/mL.
